(19) United States
(12) Patent Application Publication
(10) Pub. No.: US 2010/0163046 A1
(43) Pub. Date: Jul. 1, 2010

(54) METHOD AND APPARATUS FOR VENTILATION ASSISTANCE

(76) Inventors: Joseph Fisher, Thornhill (CA); Ludwik Fedorko, Mississauga (CA); Edward Masionis, Toronto (CA); Bryan Kowalchuk, Oakville (CA); George Volgyesi, Toronto (CA)

Correspondence Address: HERMAN & MILLMAN, 55 University Ave, Suite M002, Box 47, TORONTO, ON, M5H 3L5, CA

(21) Appl. No.: 12/294,669

(22) PCT Filed: Mar. 28, 2007
(86) PCT No.: PCT/CA2007/000497

§ 371 Date: Sep. 26, 2008

Related U.S. Application Data
(60) Provisional application No. 60/786,378, filed on Mar. 28, 2006.

(51) Int. Cl. A61M 16/00

(52) U.S. Cl. 128/204.18

(57) ABSTRACT

A CPAP device and method are provided. The CPAP device includes an inspiratory reservoir, a positive pressure air source, and a pressurizing device. The inspiratory reservoir is fluidically connectable with at least one breathing orifice of a user. The positive pressure air source is fluidically connectable to the inspiratory reservoir. The pressurizing device is movable from a first position to a second position by releasing stored potential energy. During movement to the second position the pressurizing device applies a generally constant pressure to gas contained in the inspiratory reservoir. The pressurizing device is both movable towards the first position and augmentable in stored potential energy by pressure from exhalation air from the user.

### FIELD OF THE INVENTION

The present invention relates to automatic emergency ventilatory assist devices and more particularly to a lightweight automatic emergency ventilatory assist device that can be used, for example, to assist with ventilating a patient in situ, ie. without having to transport the patient to a medical facility.

### BACKGROUND OF THE INVENTION

Pre-evacuation battlefield casualties that include respiratory distress are currently treated by rescue breathing that is administered by a soldier using mouth-to-mouth, mouth-to-nose, mouth-to-mask, or bag-valve-mask (BVM). Current emergency and transport ventilators weigh more than 12 pounds and are not completely self-contained as they usually require connection to an external pressurized gas source or external power source. The weight and size of current emergency and transport ventilators make them impractical to use for on-scene respiratory support and are more likely to be used after transport of the casualty out of the operational environment.

The ability to immediately treat respiratory distress substantially reduces the number of fatalities sustained during military operations. Civilian emergency medical technologists stress the concept of the “golden hour.” This interval represents the average time that elapses before a patient with serious or multiple injuries will begin to deteriorate rapidly. Without the ability to deliver on-scene medical support, casualties must be transported to a medical facility for treatment. This is often impossible during active operations.

Automatic emergency ventilation assistance for these casualties would eliminate the need for soldiers to be unavailable while performing rescue breathing. Treatment of these casualties in a nuclear-biological-chemical (NBC) environment is even more difficult. Casualties that occur in an NBC environment that require breathing assistance must be performed with extreme caution so as not to contaminate the casualty or the rescuer. When treating a casualty exposed to a nerve agent, it has been proposed that a cricothyroidotomy is the most practical means of providing an airway for assisted ventilation using a hand-powered ventilator equipped with an NBC filter. As part of that proposed practice, when the casualty reaches a medical treatment facility (MTF) where oxygen and a positive pressure ventilator are available, the hand-powered ventilator and NBC filter are employed continuously until adequate spontaneous respiration is resumed.

Performing a cricothyroidotomy in the field may be difficult during ongoing operations. A method to provide ventilation assistance to a casualty through an existing protective mask may save time and prevent further casualties.

Another situation facing today's Army is a chemical attack on a large group without protective masks in place. This situation may require the ventilation of hundreds of individuals making the large-scale availability of small lightweight, automatic ventilators useful.

While there are several ventilators designed for far-forward medical care, for various reasons these ventilators fall short of what is ideal for first response in the operational environment. For example, some are too heavy to be carried on foot. Some require an external source of pressurized gas or power.

In particular, urban warfare environments can create situations where fewer medics are available and difficult evacuations occur, bringing patients to relatively remote medical care. Thus it can be advantageous to have infantry that have high mobility and limited loads to carry. This rapidly deployable military force could benefit from uniquely configured medical equipment that is small, lightweight, and easily operated by available personnel. A lightweight automatic emergency ventilatory (AEV) assist device that can provide ventilatory assistance to the war fighter can enhance survivability. The size and weight of this unit will allow it to be easily transported by any soldier into urban (or other) environments. The ability for this device to operate unattended for at least an hour can allow personnel to be available for other operations, instead of providing respiratory support to a casualty using a BVM. This reduction in medical logistical load can enhance the military effectiveness.

### SUMMARY OF THE INVENTION

In a first aspect, the invention is directed to a continuous positive airway pressure CPAP device. The CPAP device includes an inspiratory reservoir, a positive pressure air source, and a pressurizing device. The inspiratory reservoir is fluidically connectable with at least one breathing orifice of a user. The positive pressure air source is fluidically connectable to the inspiratory reservoir. The pressurizing device is movable from a first position to a second position by releasing stored potential energy. During movement to the second position the pressurizing device applies a preferably generally constant pressure to gas contained in the inspiratory reservoir. The pressurizing device is both movable towards the first position and augmentable in stored potential energy by pressure from exhalation air from the user.

In a second aspect, the invention is directed to a method of assisting ventilation of a person under positive pressure, wherein the exhalation air from the person is used to assist in pressurizing the air to be inspired by the person.

In a third aspect, the invention is directed to an apparatus for assisting ventilation of a person under positive pressure, wherein the apparatus assists in carrying out the method of the second aspect.

In a fourth aspect, the invention is directed to a method of assisting ventilation of a person under positive pressure, wherein the exhalation air from the person is mixed with the air to be inspired by the person.

In a fifth aspect, the invention is directed to an apparatus for assisting ventilation of a person under positive pressure, wherein the apparatus assists in carrying out the method of the fourth aspect.

In a sixth aspect, the invention is directed to a method of assisting ventilation of a person under positive pressure, wherein an inspiratory reservoir is fed by a fan, pump or other positive pressure air source that is connected to fresh air.

In a seventh aspect, the invention is directed to an apparatus for assisting ventilation of a person under positive pressure, wherein the apparatus assists in carrying out the method of the sixth aspect.

### BRIEF DESCRIPTION OF THE DRAWINGS

The present invention will now be described by way of example only with reference to the attached drawings, in which:

FIG. 1 is a schematic view of a continuous positive airway pressure (CPAP) device in accordance with an embodiment of the present invention, showing a pressurizing device in a first position;

FIG. 2 is a schematic view of the CPAP device shown in FIG. 1, showing the pressurizing device in a second position;

FIG. 3 is a schematic view of a continuous positive airway pressure (CPAP) device in accordance with another embodiment of the present invention;

FIG. 4 is a schematic view of a continuous positive airway pressure (CPAP) device in accordance with another embodiment of the present invention; and

FIG. 5 is a schematic view of a continuous positive airway pressure (CRAP) device in accordance with another embodiment of the present invention.

### DETAILED DESCRIPTION OF THE INVENTION

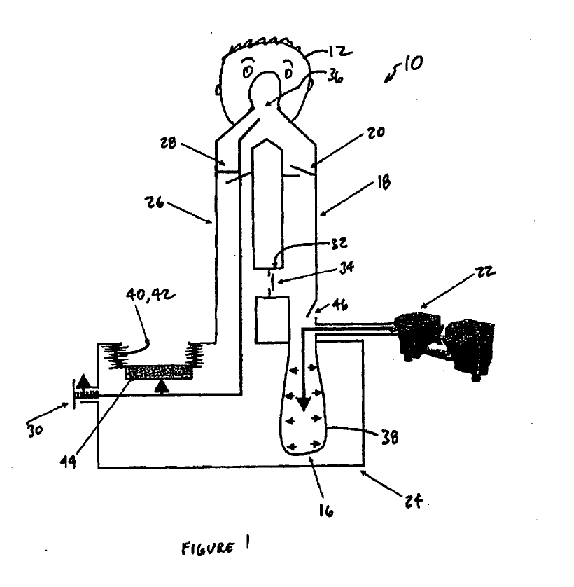

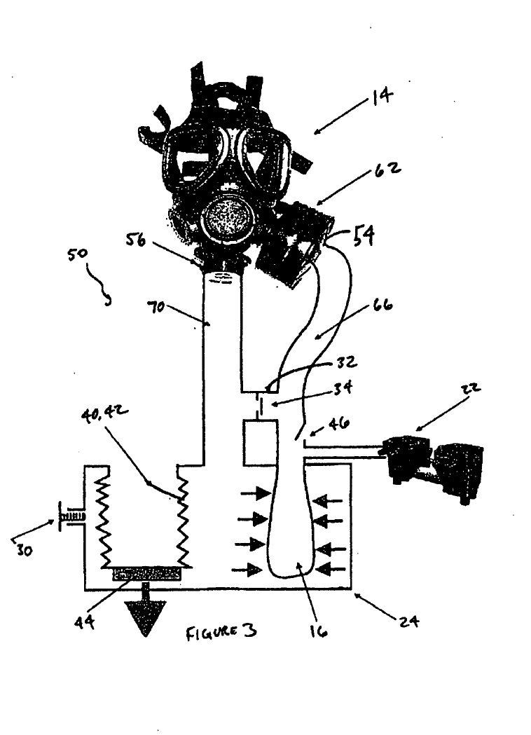

Reference is made to FIG. 1, which shows a ventilatory assist device 10 for use with a user 12 in accordance with an embodiment of the present invention. In an NBC environment, a lung injury may occur by reason of damage from a chemical or biological agent, resulting from an attack prior to the donning of a protective mask 14 (FIG. 3) by the soldier, or user 12. Once the damage has occurred, in many cases the compliance of the alveoli, (the small sacs of the lung where gas exchange occurs), may be dramatically reduced. The alveoli have a tendency to collapse on themselves after exhalation when they become smaller as a result of air leaving the lung. Once this happens, that portion of the lung becomes unusable, further reducing the soldiers ability to breathe. Furthermore, the soldier 12 will experience difficulty trying to inhale due to reduced alveolar compliance and increased lung resistance.

Both of the aforementioned problems are ameliorated by providing the soldier with positive pressure from a ventilatory assist device, such as the device 10. The positive pressure makes it easier to breathe in. When the soldier breathes out, doing so against positive pressure serves to keep the alveoli from collapsing on themselves. This mode of ventilatory assist is known as CPAP—continuous positive airway pressure.

Preferably, the CPAP device 10 would be lightweight and wearable by a soldier. For example, the CPAP device 10 could weigh less than 3 lbs including batteries if batteries are part of the device. Preferably, the CPAP device 10 would be able to provide CPAP for at least 1 hour to the soldier in case of lung damage by a chemical or biological agent. Preferably, the CPAP device 10 would be deployable by the soldier himself or herself, or by another soldier, and would interface easily with a standard gas mask.

In the embodiment shown in FIG. 1, the exhalation efforts of the soldier 12 provide a source of pressure that is captured and used to provide pressure to assist inhalation. Furthermore, the pressure is provided independently of how quickly the soldier 12 breathes.

In the embodiment shown in FIG. 1, the desired CPAP level may be, for example, approximately 10 cm H₂O of pressure. In the system shown above, the desired CPAP level is approximately 10 cm H₂O of pressure. It should be noted that while constant pressure is beneficial, a range of positive pressures may be provided as well. The CPAP device 10 includes an inspiratory reservoir 16, an inspiratory conduit 18 with an inspiratory conduit one-way valve 20, a positive pressure air source 22, an expiratory reservoir 24, an expiratory conduit 28 with an expiratory conduit one-way valve 28, a positive end expiratory pressure (PEEP) valve 30, and an optional bypass conduit 32 and valve 34. The inspiratory conduit 18 and expiratory conduit 26 are shown in FIG. 1 as meeting at a common breathing port 36 which is fluidically connected to at least one breathing orifice of the user 12. The breathing port 38 may, for example, be part of a face-mask (not shown in FIG. 1) that surrounds the nose and mouth of the user 12. It is optionally possible that the inspiratory conduit 18 and expiratory conduit 28 could each end at a separate port, namely an inspiratory conduit outlet and an expiratory conduit inlet. The two ports would be fluidically connectable to the at least one breathing orifice of the user 12.

The inspiratory reservoir 16 is fluidically connected to the one or more breathing orifices of the user 12 through the inspiratory conduit 18 and the inspiratory conduit one-way valve 20 and through the breathing port 36. The inspiratory reservoir may be defined at least in part by an inspiratory reservoir wall 38 which is flexible, and is preferably highly flexible. In a preferred embodiment, the inspiratory reservoir 16 is defined entirely by a inspiratory reservoir wall made entirely from a flexible material.

The positive pressure air source 22 is fluidically connected to the inspiratory reservoir 16 and pumps air into the reservoir 16 to maintain a selected pressure. The positive pressure air source 22 may be, for example, a pump.

The expiratory reservoir 24 is fluidically connected to the inspiratory reservoir wall 38 and preferably surrounds the entirety of the inspiratory reservoir 16. The expiratory reservoir 24 includes the PEEP valve 30 which is configured to open at a selected pressure, so that at the end of the exhalation by the user 12, the expiratory reservoir 24 has the selected pressure.

The expiratory reservoir 24 is defined at least in part by an expiratory reservoir wall 40 that is flexible. The flexible expiratory reservoir wall 40 may be, for example, a bellows 42 with a mass 44 on top, that applies a constant force and therefore a constant pressure to the flexible wall 40 and therefore to the expiratory air in the expiratory reservoir 24. The constant pressure may be any suitable amount, such as, for example, approximately 10 cm H₂O. The bellows 42 and the mass 44 together make up a pressurizing device.

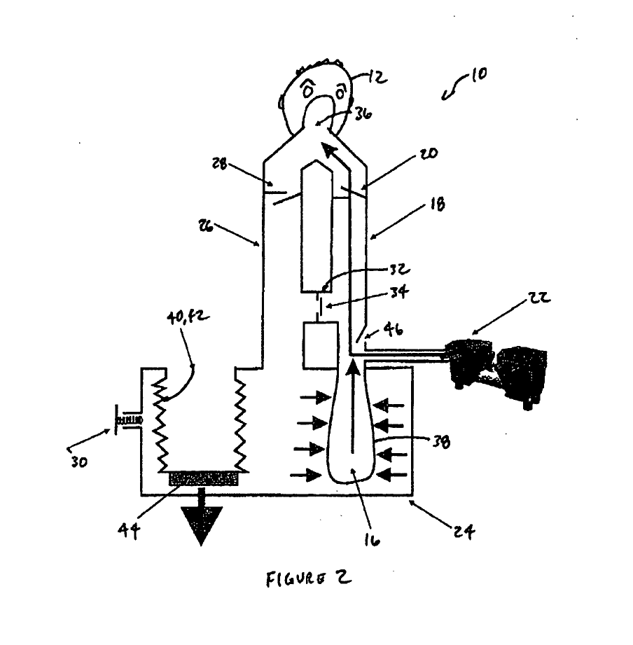

The bellows 42 is movable between a first position (FIG. 1) and a second position (FIG. 2) during the breathing cycle of the user 12. The amount of travel of the bellows 42 between its first and second positions is related to the amount of air that was exhaled by the user 12 or inspired by the user 12.

The bellows 42 has an upper limit of travel and a lower limit of travel. During exhalation by the user 12, if the bellows 42 reaches its upper limit of travel, any further exhalation increases the pressure slightly and the balance of the exhalation leaves the expiratory reservoir 24 through the PEEP valve 30.

It will be understood that the first position of the bellows 42 is not always at its upper limit of travel. The first position of the bellows 42 depends at least in part on how much air the user 12 exhales. If the user 12 does not exhale much air in a particular breath, the first position of the bellows 42, at least for that breath, may be below its upper limit of travel.

Similarly, the second position of the bellows 42 is not always at its lower limit of travel. The second position of the bellows 42 depends at least in part on how much air the user 12 inspires. If the user 12 does not inspire much air in a particular breath, the second position of the bellows 42, at least for that breath, may be above its lower limit of travel.

The bypass conduit 32 and bypass valve 34 permit the communication of expiratory air into the inspiratory conduit 18 if the inspiratory structure is unable to keep up with the instantaneous volumetric requirements of the user 12, thus depleting the inspiratory reservoir for a portion of a breath.

The PEEP valve 30 exhausts the expiratory reservoir 28 at a selected pressure, which is a little above the selected CPAP level, which may be, as noted above, 10 cm H₂O.

The device 10 functions in the following way. During exhalation, (FIG. 2) the soldier 12 breathes out through the expiratory conduit one-way valve 28. His or her exhalation fills the expiratory reservoir 24 and pushes the bellows 42 and mass 44 up. The user 12 is thus breathing out against the constant pressure provided by the bellows 42 and mass 44 of, for example, approximately 10 cm H₂O. While the user 12 exhales, the positive pressure air source 22 fills the inspiratory reservoir 16 for the next breath. For greater clarity, the air source 22 is pumping air into the inspiratory conduit 18 while the user 12 inspires, however, it is expected that the inspiratory reservoir 18 reduces in volume during inspiration. The reservoir 18 is filled by the air source 22 during exhalation.

During inspiration (FIG. 2), the soldier 12 inspires from the inspiratory reservoir 18. This reservoir 18, by virtue of being in the expiratory reservoir 24 with the bellows 42, is under constant pressure of approximately 10 cm H₂O. Thus, no matter how fast the user 12 inspires, the entire breath comes at this pressure.

Because of the presence of the inspiratory reservoir 18, the positive pressure air source 22 need not keep up with the soldiers peak inspiratory flow demand, which may be over 100 LPM. Instead the positive pressure air source only has to meet the average breathing rate which under normal breathing may be as low as 7 LPM or under walking conditions (to get himself/herself away from the front line of battle for further assistance), up to 20 LPM. The positive pressure air source 22 can therefore be much smaller and still maintain constant pressure in the system. Furthermore, because the pressure from exhalation is not wasted, but rather is used to charge the bellows for the next breath, the work of the positive pressure air source, and therefore the power and battery requirement, is reduced. Also, the average flow requirement of the positive pressure air source 22 is reduced by virtue of the fact that, if the bypass conduit 32 and bypass valve 34 are present, the positive pressure air source 22 need only provide sufficient air to ventilate the alveoli of the soldier, which is even less than his average ventilation rate. There are two reasons for this. First, average ventilation exceeds alveolar ventilation due to the requirement for part of the average ventilation being used to ventilate the trachea and other parts of the lung that do not participate in gas exchange (known as ‘dead space’). Second, alveolar ventilation requirements are determined primarily by the soldier's muscle movement, not by their breathing rate. For example, a soldier may be sitting down and hyperventilating from fear, in which case his alveolar ventilation requirements might only be 6-8 LPM (equivalent to someone sitting and not hyperventilating), even though his average ventilation from hyperventilating is 20 LPM.

An optional inspiratory relief valve 46 connected to the inspiratory conduit 18 provides a safety mechanism to permit the soldier 12 to breathe even if the positive pressure air source 22 should fail or if the reservoirs are not sufficiently large.

In FIG. 1, the bellows 42 is shown as being inside the expiratory reservoir 24. It will be appreciated, however, that the bellows 42 could alternatively be positioned out of the expiratory reservoir 24.

Reference is made to FIG. 3, which shows a device 50 in accordance with another embodiment of the present invention. As shown in FIG. 3, the device 50 may be configured to mate with a face-mask 14 that the user might already be wearing in an NBC environment. For example, the mask 14 may be an M40 mask. The mask 14 includes an inlet port 54, an outlet port 58, an inspiratory one-way valve, an expiratory one-way valve and an air fitter 62, which may be, for example, a C2 filter, for filtering air coming in through the inlet port 54.

The device 50 includes the inspiratory reservoir 16, which is connected to the mask inlet port 54 by an inspiratory conduit 68, the expiratory reservoir 24 with the PEEP valve 30, which is connected to the mask outlet port 56 by an expiratory conduit 70, an optional bypass conduit 32 and bypass one-way valve 34, the positive pressure air source 22 and the optional inspiratory relief valve 46. The device 50 may be similar to the device 10 (FIG. 1) except that the device 50 is configured to feed into the mask 14 instead of feeding directly to the one or more breathing orifices of the user 12. The device 50 would not require an inspiratory one-way valve or an expiratory one-way valve if these already exist in the mask 14.

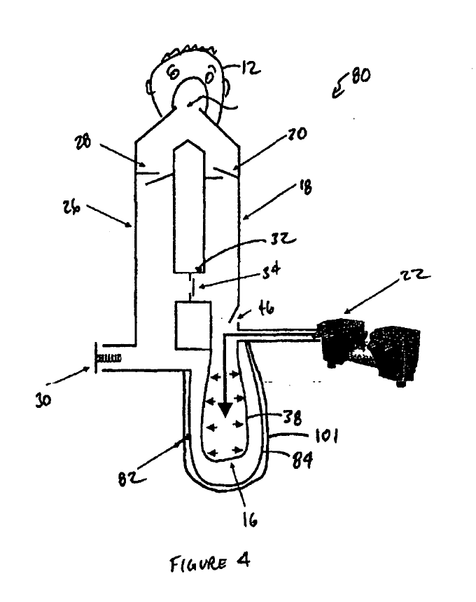

Reference is made to FIG. 4, which shows a device 80 in accordance with another embodiment of the present invention. In the embodiment shown in FIG. 4, the expiratory reservoir, shown at 82 is defined by an elastically stretchable wall 84 (eg. a balloon), instead of being defined by a rigid wan. Additionally, the expiratory reservoir 82 does not have a bellows and mass connected thereto. A non-elastic cover 101 covers the stretchable wall to prevent it from expanding indefinitely.

When the user 12 exhales, the exhalation air pressure causes the expiratory reservoir wall 84 to stretch to a first position, expanding the expiratory reservoir volume. Expansion continues during exhalation until the non-elastic cover 101 prevents the stretchable wall 84 from expanding, thus increasing the pressure in the expiratory reservoir 82 above the PEEP pressure. Any further exhalation exits the PEEP valve 30. During inspiration, the wall 84 contracts from the first position to a second position which reduces the volume of the expiratory reservoir 82, thereby increasing the pressure therein. The increased pressure is applied to the inspiratory reservoir wall 38, which urges the air contained in the inspiratory reservoir 18 towards the breathing port 36. The pressure in the expiratory reservoir 82 remains generally constant during a selected range of stretch of the reservoir wall 84. Thus, it is preferable that the first and second positions for the reservoir wall 84 be within that selected range of stretch.

The material of the reservoir wall 84 may be any suitable material, such as, for example, Latex™.

It is alternatively possible to provide a linear spring instead of a constant-pressure devices shown in FIGS. 1, 2, 3 are to provide the pressure on the gas in the expiratory gas in the expiratory reservoir. In a preferred embodiment of this alternative scenario, the linear spring could be mated to a ‘constantizing’ mechanism that converts the linear spring force to a generally constant force over a range of positions of the linear spring. For example, a linear spring could be mated to a wire that is connected to a rotatable wheel. The wheel is in turn operatively connected to a bellows or some other flexible wall on the expiratory reservoir. The wire that is connected to the wheel is connected in such a way that the force of the wire on the wheel acts at a progressively changing radius on the wheel, so that as the position of the spring changes, the radius at which the spring force acts also changes, keeping the overall torque on the wheel constant. The constant wheel torque can be converted into a constant force to operate the bellows/flexible wall on the expiratory reservoir in any suitable way.

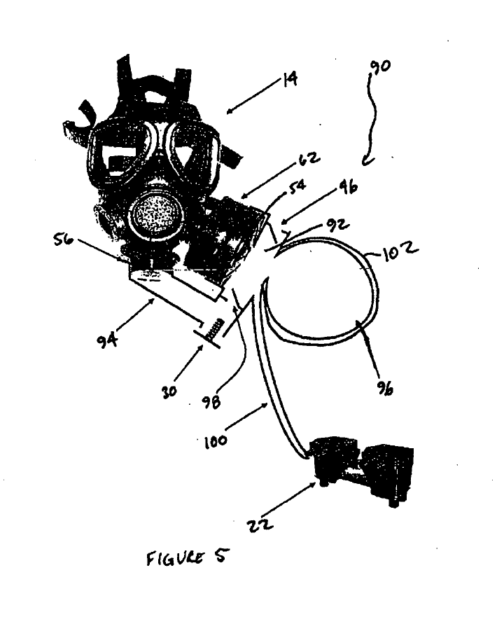

Reference is made to FIG. 5, which shows a CPAP device 90 in accordance with another embodiment of the present invention. The CPAP device 90 may be configured to operate with a mask, such as mask 14, which is equipped with one-way valves, or may alternatively be configured to have a simple mask that lacks any internal one-way valves. If a simple mask is used, then an inspiratory one-way valve, such as the inspiratory conduit one-way valve 20 shown in FIG. 1 and an expiratory conduit one-way valve such as the expiratory conduit one-way valve 28 shown in FIG. 1 may be used.

In the embodiment shown in FIG. 5, the device 90 includes an inspiratory conduit 92, the positive pressure air source 22, an expiratory conduit 94, an inspiratory reservoir 96, a one-way valve 98 at a Junction between the expiratory and inspiratory conduits 94 and 92 directed towards the inspiratory conduit 92, the PEEP valve 30 and the optional inspiratory relief valve 46.

The device 90 is configured to provide gas in the inspiratory reservoir 96 that is a combination of fresh air and expired air. The inspiratory reservoir 96 is made from an elastically stretchable material, such as Latex™ thereby permitting it to stretch and contract between first and second positions during breathing. During expiration by the user 12, the expired gas passed through the one-way valve 98 and into the inspiratory reservoir 96 along with fresh gas from the positive pressure air source 22, until the reservoir 96 expands and reaches a selected pressure, at which point the PEEP valve 30 opens to permit exhaustion of any further expired gas from the user 12.

During inspiration, the inspiratory reservoir 96 contracts and urges air contained therein towards the at least one breathing orifice of the user 12. The positive pressure air source 22 also pumps air towards the user 12.

It will be noted that the positive pressure air source 22 is connected (via conduit 100) to the section of the inspiratory structure closest to the air inlet port 54 of the mask 14. Thus, to some extent at least, the air that enters the mask initially has a higher concentration of fresh air from the positive pressure air source 22 and a lower concentration of expired air in spite of the mixing of the air from the two sources (ie. the positive pressure air source and the expiration conduit 94) that will occur in the inspiratory conduit 92.

In each of the above described embodiments, the CPAP device 10, 50, 80, 90 (shown in FIGS. 1, 3, 4 and 5 respectively) includes an inspiratory reservoir, a positive pressure air source and a pressurizing device. The inspiratory reservoir is defined at least in part by a flexible wall. In the embodiment shown in FIGS. 1, 2 and 3, the pressurizing device includes an expiratory reservoir which surrounds the inspiratory reservoir, and a mass. The expiratory reservoir includes a flexible wall that is movable between a first position and a second position wherein the expiratory reservoir has a relatively greater volume and a second position wherein the expiratory reservoir has a relatively lesser volume. The mass is connected to the flexible wall of the expiratory reservoir and has a selected weight. The weight of the mass is selected to move the flexible wall from the first position to the second position during inhalation by the user, releasing stored potential energy. During this movement to the second position, the bellows exerts a force on the expired gas in the expiratory reservoir, which, in turn, exerts a force on the inspiratory gas in the inspiratory reservoir through the flexible wall of the inspiratory reservoir, thereby urging the inspiratory gas towards the user at positive pressure. Pressure from the expired air contained in the expiratory reservoir during exhalation by the user moves the flexible wall and mass to the first position thereby augmenting their stored potential energy.

In the embodiment shown in FIG. 4, the wall of the expiratory reservoir is elastically stretchable and is movable between a first position wherein it is stretched relatively more and has a relatively greater amount of stored potential energy, and a second position wherein it is stretched relatively less, and has a relatively lower amount of stored potential energy. The pressure of the expired air in the expiratory reservoir during expiration by the user moves the expiratory reservoir wall to its first position, augmenting its stored potential energy. During inspiration by the user, the wall contracts, releasing potential energy and exerting a force on the gas in the expiratory reservoir, which in turn exerts a force on the gas in the inspiratory reservoir through the flexible wall of the inspiratory reservoir. This urges gas from the inspiratory reservoir towards the user at positive pressure. Thus the expiratory reservoir itself is part of the pressurizing device in the embodiment shown in FIG. 4.

In the embodiment shown in FIG. 5, the wall of the inspiratory reservoir is elastically stretchable and is movable between a first position wherein it is stretched relatively more and has a relatively greater amount of stored potential energy, and a second position wherein it is stretched relatively less, and has a relatively lower amount of stored potential energy. In this embodiment, expired air from the user communicates relatively directly with the inspiratory reservoir. The pressure of the expired air from the user acts on the inspiratory reservoir during expiration by the user and moves the inspiratory reservoir wall to its first position, augmenting its stored potential energy. During inspiration by the user, the inspiratory reservoir wall contracts, releasing potential energy and exerting a force on the gas therein, which urges the gas therein towards the user at positive pressure. Thus the inspiratory reservoir itself is part of the pressurizing device in the embodiment shown in FIG. 5.

Over a period of time, the instantaneous volumetric breathing rate of a user can vary substantially. If the air flow rate is to be met simply by a fan connected directly to a breathing orifice of the user, then the fan itself would have to be sized to be able to meet the instantaneous flow requirements dictated by the breathing rate of the user, which can be as high as 100 LPM in under some circumstances, or even higher. This can make the fan relatively large and heavy and generally less portable due to its weight and power consumption. By providing an inspiratory reservoir of a suitable size under constant positive pressure, the fan itself can be reduced in size and power consumption because it is only needed then to meet the average flow requirements of the user, which can be as high as 15 LPM under some circumstances, but is substantially lower than the peak instantaneous breathing rate. Thus, by providing an inspiratory reservoir, the CPAP device is relatively more portable. By additionally capturing energy from the exhalation air from the user to assist in pressurizing the inspiration air, as is described and shown for the embodiments in FIGS. 1-5, the pressure load on the fan is reduced, thereby enhancing further the portability of the CPAP device. By permitting the user to breathe in some amount of expired air, the fan no longer has to provide a flow to meet the average breathing rate by the user. Instead, the fan only has to provide sufficient flow to meet the average alveolar ventilation requirement of the user (which is lower than the average breathing rate), which further enhances the weight and power consumption characteristics of the fan and therefore the portability of the CPAP device.

It will be understood that, when the pressure being provided is called ‘constant’ or ‘generally constant’ some variability is acceptable, and is expected due to the make up of the CPAP device and its intended use. For example, the pressure can vary by plus or minus 30% or so, while still being considered generally constant and while still meeting the intended use for the device.

While the above description constitutes the preferred embodiments, it will be appreciated that the present invention is susceptible to modification and change without departing from the fair meaning of the accompanying claims.

1. A CPAP device comprising:
an inspiratory reservoir, wherein the inspiratory reservoir is fluidically connectable with at least one breathing orifice of a user;
a positive pressure air source fluidically connectable to the inspiratory reservoir; and
a pressurizing device, wherein the pressurizing device is movable from a first position to a second position wherein the pressurizing device releases stored potential energy, wherein, during movement to the second position the pressurizing device applies a generally constant pressure to gas contained in the inspiratory reservoir, wherein the pressurizing device is both movable back towards the first position and augmentable in stored potential energy by pressure from exhalation air from the user.

2. A CPAP device as claimed in claim 1, wherein the pressurizing device exerts a generally constant resistive force to pressure from the exhalation gas of the user during movement towards the first position.

3. A CPAP device as claimed in claim 1, wherein the inspiratory reservoir is defined at least in part by an inspiratory reservoir wall that is flexible, wherein the pressurizing device includes an expiratory reservoir which surrounds the inspiratory reservoir, wherein the expiratory reservoir includes a flexible wall that is movable between a first position and a second position wherein the expiratory reservoir has a relatively greater volume and a second position wherein the expiratory reservoir has a relatively lesser volume, wherein a PEEP valve is connected to the expiratory reservoir and is configured to exhaust expired gas from the expiratory reservoir to atmosphere when the pressure of gas in the expiratory reservoir reaches a selected PEEP valve opening pressure.

4. A CPAP device as claimed in claim 3, wherein a mass having a selected weight is connected to the flexible wall, wherein the weight of the mass is selected to move the flexible wall towards the second position during inhalation by the user, and wherein, in use, pressure from the exhalation air contained in the expiratory reservoir during exhalation by the user moves the flexible wall towards the first position.

5. A CPAP device as claimed in claim 3, wherein the flexible wall is elastic and is stretched elastically by a relatively greater amount when in the first position and is elastically stretched by a lesser amount when in the second position.

6. A CPAP device as claimed in claim 1, wherein the inspiratory reservoir is defined at least in part by an inspiratory reservoir wall that is flexible, wherein the pressurizing device includes an expiratory reservoir which surrounds the inspiratory reservoir, wherein the expiratory reservoir includes a flexible wall that is movable between a first position and a second position wherein the expiratory reservoir has a relatively greater volume and a second position wherein the expiratory reservoir has a relatively lesser volume, wherein a PEEP valve is connected to the expiratory reservoir and is configured to exhaust expired gas from the expiratory reservoir to atmosphere when the pressure of gas in the expiratory reservoir reaches a selected PEEP valve opening pressure, wherein the PEEP valve opening pressure controls the maximum pressure achievable in the expiratory reservoir.
